(19) United States
(12) Patent Application Publication
(10) Pub. No.: US 2022/0157451 A1
(43) Pub. Date: May 19, 2022

(54) METHODS AND SYSTEMS FOR HEALTH MANAGEMENT

(71) Applicant: IMEDGO, INC. doing business as (d.b.a.) TigoHealth, Foster City, CA (US)

(72) Inventors: Jay ELLIOT, Foster City, CA (US); Beth Anne BABER, San Diego, CA (US)

(21) Appl. No.: 17/159,030

(22) Filed: Jan. 26, 2021

Related U.S. Application Data
(60) Provisional application No. 62/966,470, filed on Jan. 27, 2020.

(51) Int. Cl. G16H 40/67; G16H 40/20; G16H 10/60; G16H 80/00; G06Q 50/26; H04M 3/51; G01S 19/01

(52) U.S. Cl. CPC G16H 40/67; G16H 40/20; G16H 10/60; G01S 19/01; G06Q 50/26; H04M 3/5116; G16H 80/00

(57) ABSTRACT

The present disclosure provides a methods and systems for providing healthcare management on a mobile electronic device of a user. A method for providing healthcare management on a mobile electronic device of a user may comprise providing the mobile electronic device comprising a health management application comprising a plurality of health management modalities. The health management application may be used to notify the user of the appointment. Upon notifying the user, the health management application may be used to provide the user with one or more transit options to permit the user to attend the medical appointment. The health management application may facilitate a connection to a care manager.

### CROSS-REFERENCE TO RELATED APPLICATIONS

This application claims the benefit of U.S. Provisional Application No. 62/966,470, filed Jan. 27, 2020, which is incorporated herein by reference in its entirety.

### BACKGROUND

Emergency room visits in the United States have risen significantly over the last decade, with rural and community-based hospitals being especially affected. While larger hospitals can have dedicated care management professionals, smaller hospitals may not have the resources to hire enough care managers to engage their patients and encourage them to seek preventative care.

Moreover, drug development and clinical trials are often slowed by a lack of access to high quality health data from a diverse population. Further, a lack of clinical trial participants can also slow the development of lifesaving drugs.

### SUMMARY

Recognized herein is a need for an integrated health management application that allows patients to access not only their medical records, but also control their medical schedules, access care managers, and facilitate attendance to medical appointments. Also recognized herein is the need for a database of health records from a diverse population that can serve as quality datasets for pharmaceutical development as well as provide possible candidates for clinical trials.

In an aspect, the present disclosure provides a method for providing healthcare management on a mobile electronic device of a user, comprising: (a) providing said mobile electronic device comprising a health management application comprising a plurality of health management modalities, wherein said plurality of health management modalities comprises (i) a records modality that provides a health record of said user from a health records database, (ii) an appointment modality that manages a medical appointment of said user with a medical provider with aid of a healthcare worker that is different than said medical provider and (iii) a care manager modality that facilitates communication between said user and a care manager of said user; (b) using said health management application to notify said user of said appointment; and (c) upon notifying said user, using said health management application to provide said user with one or more transit options to permit said user to attend said medical appointment.

In some embodiments, the method further comprises, prior to (a), creating a user profile for said user. In some embodiments, said mobile electronic device comprises an operating system that executes said health management application. In some embodiments, the method further comprises providing a one-touch emergency response system to said user. In some embodiments, said one-touch emergency response system determines a location of said user with an accuracy of less than about 1 meter. In some embodiments, said one-touch emergency response system transmits information to a call center. In some embodiments, said call center contacts emergency medical services near said user. In some embodiments, said information comprises textual information. In some embodiments, said information does not comprise a voice information. In some embodiments, said call center has access to said one or more health records of said user. In some embodiments, said one-touch emergency response system determines a location of said user within less than about 1 second. In some embodiments, said user is a recipient of healthcare from a governing body. In some embodiments, said managing is managing performed by one or more people who are not said user. In some embodiments, the method further comprises (iii) a care manager modality that facilitates communication between said user and a care manager of said user. In some embodiments, said healthcare worker is permitted to access said health record.

In another aspect, the present disclosure provides a method for generating health datasets, comprising: (a) retrieving health information of a plurality of users, wherein said plurality of users are users of a mobile healthcare application platform comprising a plurality of mobile devices of said plurality of users, and wherein at least a portion of said health information is retrieved through said mobile healthcare application platform; (b) segmenting said health information into a plurality of health datasets, wherein a health dataset of said plurality of health datasets is associated with a party among a plurality of parties, wherein said plurality of parties is different than said plurality of users; and (c) transmitting said health dataset to said party.

In some embodiments, said at least said portion of said health information is retrieved through said plurality of mobile devices. In some embodiments, said segmenting comprises segmenting using one or more machine learning algorithms. In some embodiments, the method further comprises contacting at least one user of said plurality of users regarding participation in one or more clinical trials. In some embodiments, the method further comprises applying a trained machine learning algorithm to said health dataset. In some embodiments, said health information comprises social determinants of health data.

In another aspect, the present disclosure provides a system for providing healthcare management, comprising: computer memory comprising a health management application that upon execution comprises a plurality of health management modalities, wherein said plurality of health management modalities comprises (i) a records modality that provides a health record of said user from a health records database, (ii) an appointment modality that manages a medical appointment of said user with a medical provider with aid of a healthcare worker that is different than said medical provider, and (iii) a care manager modality that facilitates communication between said user and a care manager of said user; and one or more computer processors operatively coupled to said computer memory, wherein said one or more computer processors are individually or collectively programmed to (i) use said health management application to notify said user of said appointment; and (ii) upon notifying said user, use said health management application to provide said user with one or more transit options to permit said user to attend said medical appointment.

In another aspect, the present disclosure provides a system for generating health datasets, comprising: a database comprising health information of a plurality of users; and one or more computer processors operatively coupled to said memory, wherein said one or more computer processors are individually or collectively programmed to (i) retrieve said health information of a plurality of users from said database, wherein said plurality of users are users of a mobile healthcare application platform comprising a plurality of mobile devices of said plurality of users, and wherein at least a portion of said health information is retrieved through said mobile healthcare application platform; (ii) segment said health information into a plurality of health datasets, wherein a health dataset of said plurality of health datasets is associated with a party among a plurality of parties, wherein said plurality of parties is different than said plurality of users; and (iii) transmit said health dataset to said party.

In another aspect, the present disclosure provides a non-transitory computer-readable medium comprising machine-executable code that, upon execution by one or more computer processors, implements a method for providing healthcare management on a mobile electronic device of a user, said mobile electronic device comprising a health management application comprising a plurality of health management modalities, wherein said plurality of health management modalities comprises (i) a records modality that provides a health record of said user from a health records database, (ii) an appointment modality that manages a medical appointment of said user with a medical provider with aid of a healthcare worker that is different than said medical provider, and (iii) a care manager modality that facilitates communication between said user and a care manager of said user, wherein said method comprises: (a) using said health management application to notify said user of said appointment; and (b) upon notifying said user, using said health management application to provide said user with one or more transit options to permit said user to attend said medical appointment.

In another aspect, the present disclosure provides a non-transitory computer-readable medium comprising machine-executable code that, upon execution by one or more computer processors, implements a method for generating health datasets, said method comprising: (a) retrieving health information of a plurality of users, wherein said plurality of users are users of a mobile healthcare application platform comprising a plurality of mobile devices of said plurality of users, and wherein at least a portion of said health information is retrieved through said mobile healthcare application platform; (b) segmenting said health information into a plurality of health datasets, wherein a health dataset of said plurality of health datasets is associated with a party among a plurality of parties, wherein said plurality of parties is different than said plurality of users; and (c) transmitting said health dataset to said party.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 schematically illustrates a plurality of modalities of a health management application in accordance with some embodiments.

FIG. 2 is a flow chart of an example process for an emergency response modality in accordance with some embodiments.

FIG. 3 shows an example user interface comprising multiple modalities in accordance with some embodiments.

FIG. 4 schematically illustrates a process for generating health datasets in accordance with some embodiments.

FIG. 5 is a flow chart of an example process for generating health datasets in accordance with some embodiments.

FIG. 6 shows a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “health record,” as used herein, generally refers to information related to a person's health. A health record may be a record from a doctor, a dentist, a psychiatrist, and the like. A health record may be information input by a person (e.g., self-reported symptoms). A health record may be a digital health record.

The term “modality,” as used herein, generally refers to functional subunit. A modality may be a subunit of a method, a system, a non-transitory computer readable medium comprising machine-executable code, or the like. A modality may have a specified function (e.g., controlling access to records, providing contact with a care worker). A modality may comprise a plurality of specified functions (e.g., managing a plurality of transit options to and from an appointment). A modality may operate in conjunction or in a synergistic fashion with one or more other modalities. For example, a care manager modality can interface with an appointments modality. Alternatively, a modality may operate in a stand-alone fashion.

The term “care manager,” as used herein, generally refers to a person who manages at least an aspect of the healthcare of one or more users. The care manager may be an administrator (e.g., an administrator at a healthcare facility), a proxy (e.g., a person who makes healthcare decisions for one or more users), or an authorized advocate (e.g., a living trust, a hired healthcare advocate). The care manager may be a hired worker who assists in managing healthcare scheduling and/or records of a plurality of users (e.g., a worker who manages care for a hospital). The care manager may be a nurse (e.g., a personal nurse). The care manager may be a family member (e.g., a family member tasked with managing care for a user). The care manager may schedule care for the user (e.g., schedule doctor's appointments), review healthcare records of the user (e.g., review recent results from a physician's visit), make some healthcare decisions for the user (e.g., plan follow up appointments for the user), act on behalf of and for the user (e.g., interface with insurance for the user, or any combination thereof. The care manager may be different from a medical provider. The care manager may be different than a healthcare worker. The care manager may be employed by a hospital, an insurance company, a medical center, or the like.

In an aspect, the present disclosure provides methods for providing healthcare management on mobile electronic devices. A method for providing healthcare management on a mobile electronic device of a user may comprise providing the mobile electronic device comprising a health management application comprising a plurality of health management modalities. The plurality of health management modalities may comprise (i) a records modality that provides a health record of the user from a health records database, and (ii) an appointment modality that manages a medical appointment of the user with a medical provider with aid of a healthcare worker that is different than the medical provider. The healthcare worker may be permitted to access the health record. Next, the health management application may be used to notify the user of the appointment. Next, upon notifying the user, the health management application may be used to provide the user with one or more transit options to permit the user to attend the medical appointment.

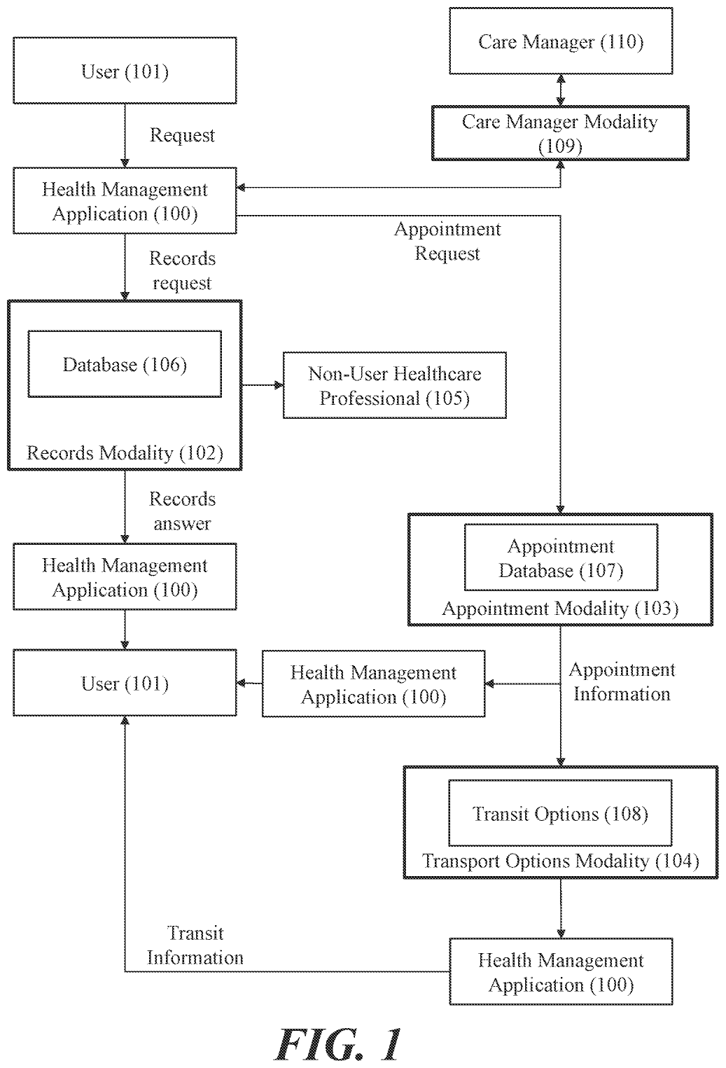

FIG. 1 schematically illustrates a plurality of modalities of a health management application 100. The application 100 may be implemented on one or more appropriately programmed or configured computers or computer systems in one or more places. The application 100 may comprise at least 1, 2, 3, 4, 5, 6, 7, 8, 9, 10, or more modalities. The modalities may operate independently of each other. As an alternative, at least some of the modalities may operate synergistically. The application may comprise a records modality 102, an appointment modality 103, a care manager modality 110, and/or a transit options modality 104. The records modality may receive a records request from a user 101 using the healthcare application 100. The records request may be a request by the user for their own medical records. Alternatively, the records request may be for medical records of another person (e.g., a spouse, a child, etc.). The user may request records to be sent to one or more non-user healthcare professionals 105. For example, a user can request that the results of a blood test be sent to their primary care physician and their spouse. The records modality 102 may comprise a database 106. The database may be a database that complies with one or more government regulations. For example, the database can be a Health Insurance Portability and Accountability Act (HIPAA) compliant database. The database may be located on one or more distributed computing systems (e.g., a cloud infrastructure). The database may be stored in local storage on a device of the user. At least a portion of the database may be both stored on a device of the user and on a distributed computing system. For example, the user can maintain records of their last checkup on their mobile device while their full medical history is maintained on a distributed computing system. The health management application 100 may process the records request from the user 101, which may include retrieving one or more health records from the database 106 and displaying the records to the user via the health management application. In an example, a user may visit a new doctor who may ask about any past procedures. In this example, the user can use the health management application 100 to access their health records to show the doctor in real time, as well as permit the new doctor to access the medical records in the future by giving permission through the health management application.

The records modality may further comprise data related to one or more social determinants of health (SDH) data. The SDH data may comprise user data related to one or more of the following: food security, transportation options available, housing security, the local environment where a user lives and/or works (e.g., pollution data, crime statistics, allergen concentrations, water quality, etc.), education, access to healthcare, health literacy, work conditions (e.g., workplace safety, employment status, job security), social connectedness, social support structures, culture, history of incarceration, social status, history of addiction, citizenship status, stress levels, income, race, housing status, or any combination thereof. The SDH data may be combined with the one or more health records of the user. The combined SDH data and one or more health records of the user may be input into a machine learning algorithm. The machine learning algorithm may be configured to process the combined data to predict one or more needs of the user. The machine learning algorithm may be configured to reduce the number of emergency room visits by the user by identifying potential issues early. For example, a user living in temporary housing with food insecurity and a history of diabetes can be at a higher risk for a severe diabetic episode. In this example, the machine learning algorithm can identify the increased risk and alert a patient care manager to schedule an appointment with the user's primary care physician. The SDH data may be gathered from one or more interviews with the user or one or more persons associated with the user (e.g., providing the user with a questionnaire, asking questions of the user's family members), one or more observations of a medical provider of the user (e.g., a doctor's note), one or more demographic surveys (e.g., census data, data derived from a survey given by another organization), medical metadata (e.g., nutrient levels over time in blood tests of the user), or the like, or any combination thereof.

The appointment modality 103 may be configured to receive an appointment request from the user 101 via the health management application 100. The appointment modality 103 may manage a medical appointment of the user with a medical provider. For example, a physician instructs the user to return after three months for a follow-up consultation and the user inputs the date into the appointment modality to block of time on the user's calendar and remind the user of the appointment. The appointment modality may manage the appointment of the user with aid of one or more healthcare workers. The one or more healthcare workers may be different than the medical provider. The one or more healthcare workers may be associated with the medical provider. For example, a physician instructs their assistant to schedule a follow-up appointment with a user. In this example, the assistant can interface directly with the appointment modality, allowing the assistant to generate an appointment for the user, remind the user of the appointment, and update the appointment as needed. In another example, a care manager at a hospital can reschedule a missed appointment for a user. The healthcare worker may be an employee of the medical provider. The healthcare worker may have access to the health records of the user. The healthcare worker may be permitted to access the health records of the user. For example, a user can give permission to a physician's assistant to access the health records of the user to facilitate follow-up with the user. The healthcare worker may have access to the health records of the user though the medical provider. The healthcare worker may not have access through the records modality. For example, the healthcare worker can have access to the user's health records through the healthcare provider, but not through the records modality. The appointment modality may comprise an appointment database 107. The appointment database 107 may be configured to comprise information about the appointments of the users of the health management application. For example, the appointment database can comprise data such as the number of appointments made for a plurality of users, the attendance rate of those appointments, and the weather for the days those appointments were on and provide a basis to analyze if there is a correlation between the attendance of the users and the weather. The appointment database may provide data to one or more computer processors configured to analyze the data of the database for one or more trends. The one or more computer processors may analyze data not contained within the appointment database. The analysis of the data may generate information used to improve future appointments. For example, if a user consistently misses appointments scheduled for Tuesday, the appointment modality can recommend not creating new appointments on Tuesdays.

The appointment modality may be configured to facilitate communication directly between the user and a scheduling system used by a care provider. The scheduling system may be a live scheduler (e.g., a person who schedules appointments), an automated scheduling system (e.g., a telephone tree based scheduler, an online scheduler, etc.), or a combination thereof. The appointment modality may be configured to interface with a scheduling system and extract scheduled appointments. The appointment modality may be configured to generate reminders for the user regarding the scheduled appointments. The reminder may be an alarm, a push notification, an email, a call, or the like, or any combination thereof. For example, the appointment modality can retrieve an appointment made by a user with their pediatrist, provide a calendar event on the user's calendar, and remind the user via a push notification to the user's phone the day before the appointment. The appointment modality may be configured to facilitate communication between the user and the scheduling system if the user needs to reschedule the appointment. The appointment modality may be configured to recognize potential scheduling conflicts and notify the user. The appointment modality may be configured to automatically attempt to reschedule an appointment if a conflict arises. The appointment modality may use a machine learning algorithm as described elsewhere herein to determine that an appointment is to be rescheduled and/or reschedule the appointment. The appointment modality may be configured to simply facilitate contact between the user and the scheduling system. The appointment modality may access user appointment information from a database maintained by the medical provider. For example, the appointment modality can request all upcoming appointments for a given user that are scheduled using the medical provider's own appointment system. The appointment modality may not send new data to the medical provider's scheduling system. For example, the appointment modality can download current appointments but not upload new appointments.

The appointment modality may comprise a machine learning algorithm. The machine learning algorithm may be configured to analyze the data of the database as described above. The machine learning algorithm may be configured to determine trends in the data of the appointment database. The machine learning algorithm may be configured to provide user management functionalities. The user management functionalities may be scheduling user appointments, reminding users of upcoming appointments, interfacing with user managers (e.g., assisting patient care workers), assisting users with payments, generating user statistics (e.g., on time percentages), or the like. The machine learning algorithm may interface with the user. For example, the machine learning algorithm can power a chatbot that a user can input symptoms into, and the algorithm can process those symptoms to determine how quickly the user should visit a doctor. In this example, the machine learning algorithm can interface with the user's calendar and the doctor's calendar to schedule an appointment, taking into account historical factors of the user's appointment attendance. In another example, the user can direct the machine learning algorithm to find a time in the next week for the user to have a blood test. In this example, the machine learning algorithm can review the user's schedule, as well as the schedule of the blood test facility, and select an appropriate time for the test. The machine learning algorithm may interface with an appointment system of a medical provider, a health records database of a provider, a health records database not of a provider, or any combination thereof.

The transport options modality 104 may receive appointment information from the appointments modality 103. The transport options modality may use the appointment information to generate one or more transit options 108. The one or more transit options may be walking directions, human powered device directions (e.g., bicycle directions, skateboard directions), driving directions, public transit directions (e.g., busses, trains), ride share options (e.g., Lyft®, Uber®, taxi services), shared device options (e.g., bicycle share programs, shared scooters), non-emergency medical transport services, and the like. The transport options modality may comprise a machine learning algorithm. The machine learning algorithm may be configured to process data from the appointment database 107 and a history of the user's transit utilization. The machine learning algorithm may be configured to select transit options that provide the highest likelihood for the user to attend an appointment.

The transport options modality may present the one or more transit options to the user 101. The user may select a transit option from the one or more transition options. The transport options modality may be configurable to the preferences of the user. The transport options modality may be configured to book a transport option on behalf of the user. For example, a user has indicated that they prefer taking the train to attend their doctor's appointments, so the transport options modality purchases a train ticket for the user to attend an upcoming doctor's appointment. In this example, the transport options modality can purchase the ticket for a train that leaves 15 minutes earlier than is necessary for the user to get to the doctor's appointment because the user has a history of arriving 15 minutes late.

The health management application may be configured to permit a user to create a profile with the health management application. The profile may be generated by the user (e.g., provided by the user when the user signs up for the health management application) or generated by a person other than the user (e.g., a healthcare care manager). For example, a care manager at a hospital can generate user accounts for each patient of the hospital. The profile may comprise information about the user (e.g., name, address), health information about the user (e.g., the user is diabetic), preferences of the user (e.g., the user prefers the application to be in Spanish), and the like. The profile may enable the user to save information on the healthcare application. For example, a user can save to their profile that they are a hemophiliac, and the information can be immediately displayed to an emergency responder accessing the profile.

The health management application may be configured to be run on a mobile electronic device. The mobile electronic device may be a smartphone. The mobile electronic device may comprise an operating system that executes the health management application. For example, the health application can be a single mobile application comprising the one or more modalities that is executed on the operating system. The operating system may be Android®, iOS®, iPadOS®, watchOS®, Windows Phone®, Tizen®, KaiOS®, or the like.

The user may be a recipient of healthcare from a governing body. Each user of the health management application may be a recipient of healthcare from a governing body. Examples of healthcare from a governing body comprise Medicaid, Medicare, children's health insurance program (CHIP), state health insurance assistance program (SHIP), Medi-Cal, the National Health Service (NHS), other national health insurance programs, and the like. The health management application may be configured to efficiently interface with a governing body run healthcare program. For example, the health management application can be configured to display the appropriate reimbursement codes for Medi-Cal. The health management application may improve a quality of care provided by the healthcare program. For example, better patient management at a lower cost can reduce the number of expensive emergency room visits by recipients of Medicare. The health management application may be provided by a governing body run healthcare program. For example, the NHS can provide the mobile healthcare application to its recipients.

The managing may be performed by one or more people who are not the user. The managing may be managing of health records, user appointments, access to a care manager, user transportation options, or the like, or any combination thereof. A computer algorithm may perform the managing. The computer algorithm may be a machine learning algorithm. The managing may comprise scheduling appointments, performing post-appointment follow-ups, assisting the user with attending an appointment, providing access to one or more healthcare records, other patient checkup activities, or any combination thereof. For example, a healthcare professional can utilize the health management application to schedule an appointment for a user in response to a doctor requesting a follow-up. The managing may be performed on behalf of a medical center (e.g., doctor's office, hospital).

The health management application may be configured to be used by a medical center to manage a user who is a patient of the medical center. The health management application may permit a worker of the medical center to interface with the user. For example, the health management application can have a chat functionality whereby the worker can contact the user to quickly clarify an issue with the user's insurance. The health management application may further comprise a care manager modality 109. The care manager modality may be configured to facilitate direct contact between a user 101 and a care management professional 110 assigned to the user. The care management professional may be employed by a medical provider, a payer (e.g., private insurance, Medicare, etc.), the user, or any combination thereof. The user and the care management professional may communicate via a text interface (e.g., SMS texting, instant messaging, e-mail, etc.), a vocal interface (e.g., a one-touch telephone call), a video interface (e.g., a video chat session), or any combination thereof. For example, a user can press a button on the screen of a smartphone app that servers as a user interface for the health management application and initiate a telephone call with the user's care manager. In this example, the care manager can advise the user of the importance of an upcoming appointment. The user may initiate contact with the care manager. Alternatively, the care manager may initiate contact with the user. For example, a care manager can initiate a chat session with the user to follow up on a recent doctor's visit by the user. The care manager modality may be configured to direct the user to the appropriate care. For example, a user with a minor sprain can contact a care manager via the care manager modality, and the care manager can divert the user from going to the emergency room and instead direct the user to go to an urgent care clinic. In another example, a user experiencing a retinal detachment can contact the care manager via the care manager modality. In this example, the care manager can make an emergency appointment for the user with an optician. The care manager modality may be configured to facilitate a care manager to request an authorization for a user to meet with a specialist. The care manager may have access to the health records of the user (e.g., via the health records modality, via the provider's records system). The care manager may have access to an appointment system of the medical provider. The appointment system of the medical provider may be different than the appointment modality.

The health management application can improve the ability of the medical center to interface with the user, and thus improve the efficiency and quality of care provided. The health management application may be configured to manage records generated by a medical center. For example, a worker of the medical center can input new test results into a database managed by the health management application. In this example, the database can make the results available to the user and all of the user's other healthcare providers quickly and efficiently. In another example, a user can permit a new doctor to access the user's healthcare records online. In another example, a worker of the medical center can input new test results into the medical center's database, and the health management application can access the medical center's database, download the new test results, and make the results available to the user.

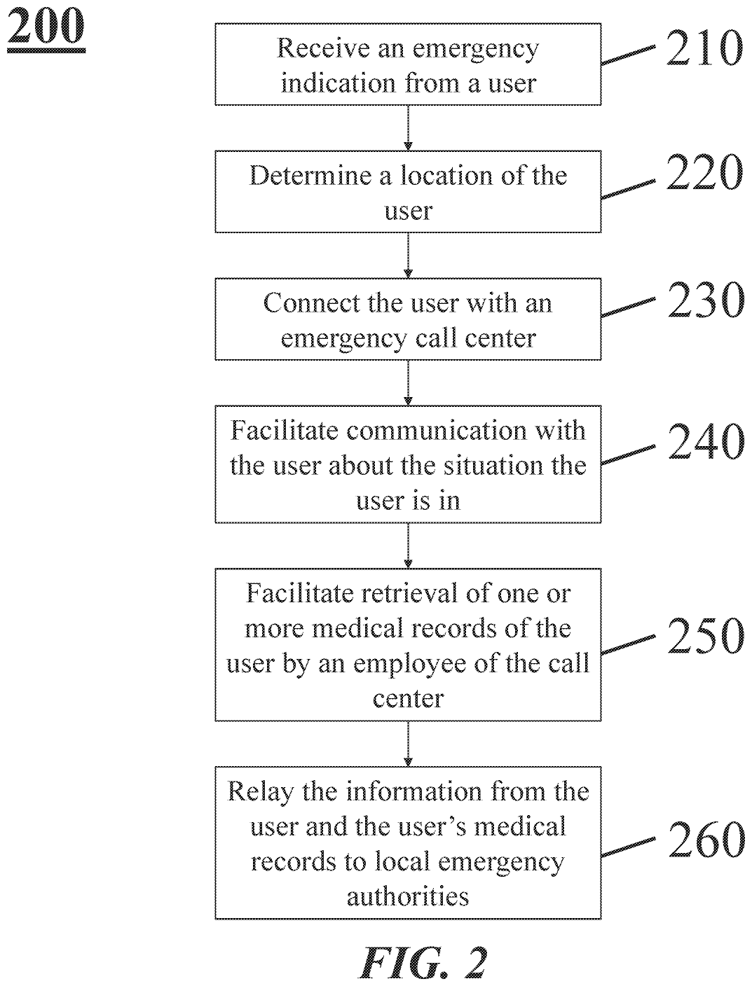

FIG. 2 is a flow chart of an example process for an emergency response modality. The process can be performed by the health management application 100 of FIG. 1, which may be implemented on one or more appropriately-programmed computers in one or more locations. For example, the process 200 can be performed by one or more servers that host the health management application and one or more user devices (e.g., mobile device or laptop computers) that run user-instances of the health management application. The health management application may receive an emergency indication from a user (210). The emergency indication may be based at least in part on an interaction of the user with the health management application. The interaction may be pressing a button (e.g., on a screen of a device, a physical button of a device), a vocal command, device sensor data (e.g., a change in the reading of an accelerometer in the device), an indication to a camera of a device, any combination thereof, or the like. For example, a user in distress can open the health management application and perform a three second press on a virtual button in the health management application. The emergency indication may be a multi-step indication. For example, a user can press one button on the screen of the user's device, and then swipe an indicator across the screen of the device. The emergency indication may comprise recording one or more of audio data, photographic data, videographic data, and the like. The photographic and/or vidoegraphic data may be taken using at least one front camera of the device (e.g., a selfie camera), at least one rear camera of the device, or a combination thereof. For example, when a user activates an emergency indication, the user's phone can take a picture of the user. The recordation of additional data may improve the preparedness of an emergency responder responding to the emergency indication. The emergency indication may comprise one or more indications of a status of the user. The status of the user may comprise information such as consciousness state, injury state, presence of an altered mind, and the like. The emergency response modality may further comprise a list of emergency contacts. The list of emergency contacts may be input by the user. The emergency contacts may be dynamic (e.g., adjustable by the user) or fixed (e.g., predetermined and not changeable). The list of emergency contacts may be a list of at least about 1, 2, 3, 4, 5, 6, 7, 8, 9, 10, 11, 12, 13, 14, 15, 16, 17, 18, 19, 20, or more contacts. The list of emergency contacts may be a list of 20, 19, 18, 17, 16, 15, 14, 13, 12, 11, 10, 9, 8, 7, 6, 5, 4, 3, 2, 1, or less contacts. The contacts may be personal relations (e.g., family, friends), business relations (e.g., managers), medical professionals (e.g., primary care physicians), or any combination thereof. The emergency response modality may send a notification at least a subset of the emergency contacts upon receiving an emergency indication from the user. The notification may comprise an indication of an emergency (e.g., a message stating an emergency indication was received), a location of the user (e.g., the GPS location of the user when the emergency indication was received), information on the condition of the user (e.g., speed the user was traveling, an image recorded by the emergency response modality), or any combination thereof. The notification may be a text message (e.g., SMS message), an email, a push notification, a telephone call, or any combination thereof. The notification may comprise a link to another application. For example, a notification can include a link to open the user's location in a maps application.

The system may determine a location of the user (220). The location of the user may be determined by one or more of the cellular network position of the user, cellular network strength at the location of the user, the global positioning system (GPS) location of the user, environmental indications around the user (e.g., sounds, visible objects and landmarks, etc.), sensor data of the device of the user (e.g., compass direction, device orientation), device location services (e.g., nearby Bluetooth® signals), or any combination thereof. The location of the user may be determined within at least about 0.1 meters (m), 0.5 m, 1 m, 1.5 m, 2 m, 2.5 m, 3 m, 4 m, 5 m, 10 m, 25 m, 50 m, or more. The location of the user may be determined within at most about 50 m, 25 m, 10 m, 5 m, 4 m, 3 m, 2.5 m, 2 m, 1.5 m, 1 m, 0.5 m, 0.1 m, or less. The location of the user may be determined within at least about 0.1 seconds (s), 0.5 s, 1 s, 2 s, 3 s, 4 s, 5 s, 6 s, 7 s, 8 s, 9 s, 10 s, 15 s, 30 s, 45 s, 60 s, 120 s, 180 s, or more. The location of the user may be determined within at most about 180 s, 120 s, 60 s, 45 s, 30 s, 15 s, 10 s, 9 s, 8 s, 7 s, 6 s, 5 s, 4 s, 3 s, 2 s, 1 s, 0.5 s, 0.1 s, or less. For example, a user can press an emergency alert button on the screen of a smartphone, and within 5 seconds the emergency response modality can determine the user's location with an accuracy of 1 meter. The emergency response modality may be configured to determine if a user is moving. The emergency response modality may determine a user is moving using data gathered from a mobile device of the user. The data may comprise device data (e.g., accelerometer data, compass data), position data (e.g., cellular network position, global positioning system location), sensor data (e.g., camera data, audio data), or any combination thereof. If the emergency response modality determines it is likely a user is moving, the emergency response modality may update the location of the user. The emergency response modality may update the location of the user every at least about 0.5, 1, 2, 3, 4, 5, 6, 7, 8, 9, 10, 11, 12, 13, 14, 15, 16, 17, 18, 19, 20, 30, 40, 50, 60, or more seconds. The emergency response modality may update the location of the user every at most about 60, 50, 40, 30, 20, 19, 18, 17, 16, 15, 14, 13, 12, 11, 10, 9, 8, 7, 6, 5, 4, 3, 2, 1, 0.5 or less seconds. The updating the location of the user may further comprise updating a speed of the user, a direction the user is traveling, a location history of the user, and the like.

The system may connect the user with an emergency call center (230). The emergency call center may be different than the emergency medical services (EMS). The emergency call center may be in a location other than the location of the user (e.g., in another state). The user may be connected to a human operator or a non-human operator (e.g., a computer program) within the emergency call center. The user may be connected to the emergency call center within at least about 0.5 s, 1 s, 2 s, 3 s, 4 s, 5 s, 6 s, 7 s, 8 s, 9 s, 10 s, 15 s, 30 s, 45 s, 60 s, 120 s, 180 s, or more. The user may be connected to the emergency call center within at most about 180 s, 120 s, 60 s, 45 s, 30 s, 15 s, 10 s, 9 s, 8 s, 7 s, 6 s, 5 s, 4 s, 3 s, 2 s, 1 s, 0.5 s, or less. The user may be connected to the emergency call center using a text message, a voice call, a video call, or any combination thereof. The video call may be a one way or a two-way video call. The emergency call center may be configured to receive non-audio emergency indications. For example, a worker at the emergency call center can have access to a messaging application to have a text conversation with a user in an emergency. The emergency call center may have control over the device the user uses to connect with the emergency call center. The control may be a control over an audio detection device (e.g., a microphone), an image detection device (e.g., a camera), a user input device (e.g., a touchscreen, a device mounted radio detection and ranging (RADAR) or light detection and ranging (LIDAR) system), or any combination thereof. For example, an emergency call center worker can activate the front and rear cameras of a user's cell phone to assess the situation the user is in and determine environmental hazards around the user.

The system may facilitate communication with the user about the situation the user is in (240). The communication may be via the connection of the user and the emergency call center. For example, a user connected by an audio call to the emergency call center can speak with an employee of the emergency call center. The facilitation may comprise reconnecting a user and an emergency call center if the initial connection between the user and the emergency call center is disrupted. For example, a user connects to a call center via a video call that drops due to a poor network connection. In this example, the system can reinstate the call between the user and the emergency call center and disable the video to improve the stability of the call. The system may be configured to control the device the user initiates the contact with the emergency call center on. For example, a user initiates via a smartphone a text-based emergency session with an emergency call center and communicates with the call center for five minutes before no longer responding. In this example, the emergency call center can initiate audio and video recording using the user's phone to determine the status of the user and if the user is still conscious. The user may be able to transmit one or more messages, images, videos, or any combination thereof to the emergency call center to improve the understanding of the user's emergency. For example, a user with a broken arm can take a series of images of the break and send the images to the emergency call center. The system may be configured to permit the user and the emergency call center operator to communicate in a way different that the initial communication. For example, a user who initially made an audio call to the emergency call center can enable video on the same call.

The system may facilitate retrieval of one or more medical records of the user by an employee of the call center (250). The emergency call center may have access to the medical records of the user. The emergency call center may be given access to one or more medical records of the user by the user. The emergency call center may have access to a database comprising health records of the user. The emergency call center may have access to the data recorded when the emergency indication was received. For example, the emergency call center can access an image that was taken of the user when the user pressed an emergency help button on a smartphone application. The emergency call center may interact with the user. The interaction may be gathering additional information about the nature of the emergency, receiving instructions from the user (e.g., how to most effectively reach the user), providing instruction to the user, or any combination thereof. The emergency call center may have access to non-medical data related to the user. The non-medical data may comprise a picture of the user, an age of the user, biographic information about the user (e.g., name, address), the occupation of the user, and the like. The emergency call center may have access to the location of the user. The emergency call center may have access to a more precise location of the user than EMS.

The system may relay the information from the user and the user's medical records to local emergency authorities (260). The emergency call center may be configured to transmit one or more pieces of data (e.g., medical records, user images, call transcripts) to an EMS local to the user. For example, a user who broke their leg in San Francisco can be routed to a call center in Texas, where a call center worker can compile the user's medical history, images sent by the user, and the precise location of the user and send the compiled data to an EMS dispatcher in San Francisco. The emergency call center may be configured to send information about the user directly to emergency personnel (e.g., firefighters, paramedics, police officers), to emergency dispatchers, or a combination thereof.

The system may be configured to alert one or more other users of an emergency indication. For example, an alert can be sent to a user's phone when the user's mother presses an emergency indication button. The alert may comprise the time of the alert, the location of the user, the information recorded with the alert (e.g., an image), the status of the alert (e.g., the user is currently communicating with an emergency call center user), medical facilities near the user, or any combination thereof.

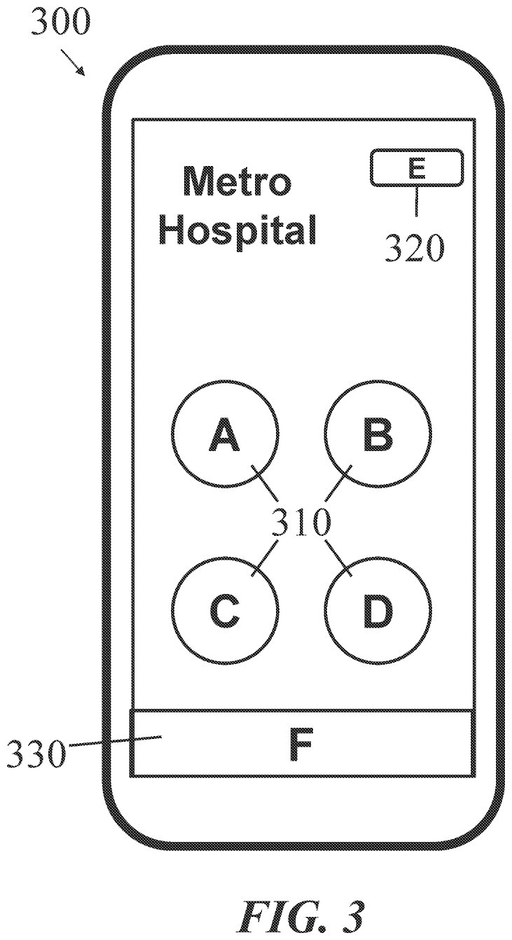

FIG. 3 shows an example user interface comprising multiple modalities. This example interface is provided as an example only to illustrate functionalities of the mobile health management platform. The functionality of the interface of the mobile health management platform is not based solely on the precise layout of the interface.

A user device 300 may comprise a screen configured to display a graphical user interface (GUI). The GUI may be configured with visual elements (e.g., buttons) 310. The GUI may have at least about 1, 2, 3, 4, 5, 6, 7, 8, 9, 10, 15, 20, 25, 30, 40, 50, or more visual elements. The visual elements may be configured to enable a user to interact with one or more modalities. The visual elements may correspond to different modalities. For example, a user can press element A to contact a care manager at a hospital, while pressing element B can navigate the user to an appointments portal where the user can schedule or revise an appointment, while pressing element C can present the user with a plurality of transit options for transport to and from a medical appointment, while pressing element D can open a navigation application to give the user directions to a medical facility at which the user has an appointment. The visual elements may be configured to open another application upon interaction by the user. The visual elements may be configurable by the user. For example, a user can change the visual elements to be shortcuts to the modalities that the user utilizes the most. Examples of modalities that the visual elements may be related to are records modalities, appointment modalities, transit options modalities, family medical modalities, and the like. The visual elements may be configured to change based at least in part on use habits of the user.

The GUI may comprise an emergency interface 320. The emergency interface may be configured to initiate the process detailed in FIG. 2. The emergency interface may comprise instructions on how to activate the emergency interface. For example, the emergency interface can read ‘Press here for three seconds to active an emergency alert.’ The emergency interface may be configured to reject accidental interactions (e.g., false presses, interactions while the device is in a pocket). The emergency interface may be at least temporarily disabled if a user registers too many false emergencies.

The GUI may comprise additional element 330. Additional element 330 may comprise navigational aids (e.g., a back button, a home button), further subsystems or modalities (e.g., quick access to medical records, access to a personalized healthcare plan), application settings (e.g., font size, color, account details), or any combination thereof.

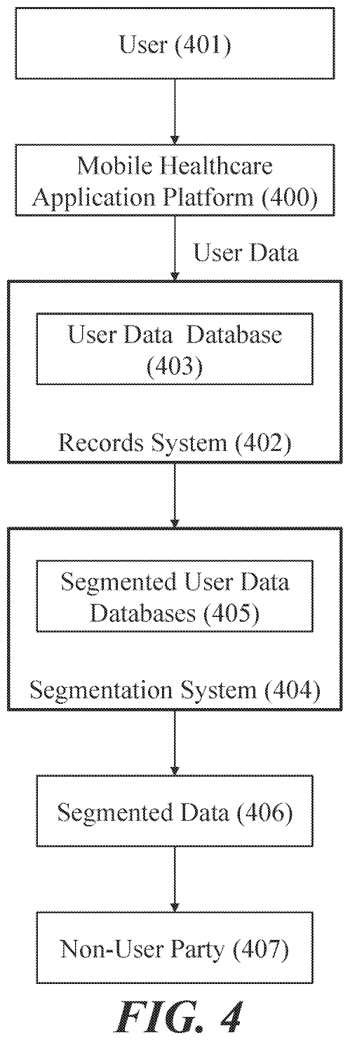

FIG. 4 schematically illustrates a process for generating health datasets. A mobile healthcare application platform 400 may be implemented on one or more appropriately programmed computers. The application platform may have one or more users 401. The user may be the same user as user 101 of FIG. 1. The user may be a different user than user 101 of FIG. 1. The users may access healthcare records and data through the application platform. The healthcare records and data may be healthcare records and data associated with the user (e.g., the user's own records). The users may give permission for a computer algorithm to access the healthcare records and data of the users. The computer algorithm may comprise a machine learning algorithm. The computer algorithm may compile the user healthcare records and data into a records system 402, which may comprise a user data database 403. The healthcare records and data may comprise biographical information (e.g., occupation, age, residency information), basic healthcare information (e.g., height, weight, blood pressure), information related to a condition of the user (e.g., diagnostic information, prognostic information), information related to a treatment the user has received (e.g., efficacy of a treatment, duration of a treatment), and the like, or any combination thereof. For example, the records and data for a user with a cancer can comprise the user's physical attributes over time, the time and duration of radiation therapies, and the results of those therapies. The healthcare records and data may comprise all available healthcare information of a user. For example, the healthcare records and data can contain a user's full medical file. The user data database may be compliant with healthcare data privacy regulations (e.g., HIPAA, European Union General Data Protection Regulation (GDPR)).

The user database may be used as a dataset for a segmentation system 404. The segmentation system may comprise a machine learning algorithm. The machine learning algorithm may be configured to analyze the user data database 403 and generate one or more segmented user data databases 405. The segmented database 405 may be segmented with regards one or more attributes of the data. For example, the segmented database can be a subset of the user data database comprising users who have a particular form of thyroid cancer. The attributes may be attributes of the healthcare records and data as described elsewhere herein.

The machine learning algorithm may further separate the segmented user data database 405 into segmented data 406. The segmented data may comprise data sharing a common attribute. For example, all data in the segmented data can be data relating to the weight of users who have a specific form of lung cancer. The segmented data may comprise all of the data related to the users whose data makes up the segmented data. For example, the segmented data can be the full medical records of patients between 25 and 35 who have received a particular chemotherapy drug. Alternatively, the segmented data may comprise a subset of the data related to the users whose data makes up the segmented data. For example, the segmented data can comprise of the ages, heights, weights, treatment regimens, and treatment outcomes of recipients of a bariatric surgery. The segmented data may be anonymized data.

The segmented data 406 may be transmitted to one or more non-user parties 407. The non-user parties may be healthcare companies, pharmaceutical companies, law firms, regulatory agencies, or the like. The non-user parties may have temporary access to the segmented data, permanent access to the segmented data, or ownership of the segmented data.

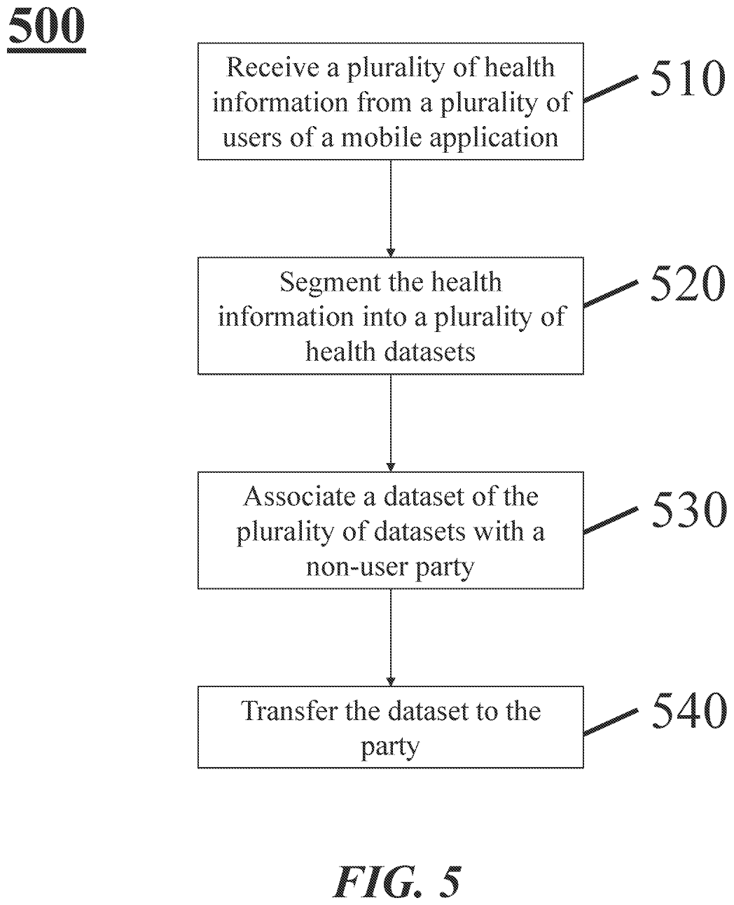

FIG. 5 is a flow chart of an example process for generating health datasets. The process can be implemented on one or more appropriately-programmed computers in one or more locations. For example, the process 500 can be performed by one or more servers that host the health management application and one or more user devices (e.g., mobile device or laptop computers) that run user-instances of the health management application. The system can receive a plurality of health information from a plurality of users of a mobile application (510). The plurality of health information may be health information provided by the plurality of users (e.g., responses to surveys, user reported symptoms) or health information provided by one or more medical facilities (e.g., doctor's notes, surgical records). The users may consent to the sharing of the plurality of health information. At least a portion of the health information may be retrieved through a plurality of mobile devices. At least a portion of the health information may be retrieved directly from one or more health information datasets (e.g., servers storing health information). The mobile application may be a mobile application as described elsewhere herein.

The system may segment the health information into a plurality of health datasets (520). One or more machine learning algorithms may perform the segmentation. The machine learning algorithm may be a trained machine learning algorithm. The machine learning algorithm may be a clustering algorithm. The clustering algorithm may be a centroid-based clustering algorithm (e.g., a k-means algorithm), a density-based clustering algorithm, a distribution-based clustering algorithm, a hierarchical clustering algorithm, or the like. The machine learning algorithm may segment the health information into at least about 2, 3, 4, 5, 10, 25, 50, 75, 100, 250, 500, 1,000, 2,500, 5,000, 10,000, 50,000, 100,000, 250,000, 500,000, 1,000,000, or more segments. The machine learning algorithm may segment the health information into at most about 1,000,000, 500,000, 250,000, 100,000, 50,000, 10,000, 5,000, 2,500, 1,000, 500, 250, 100, 75, 50, 25, 10, 5, 4, 3, 2, or less segments. The machine learning algorithm may segment the health information based at least in part on one or more attributes of the health information and/or attributes of the users the health information is derived from. The machine learning algorithm may be given instruction on the segments to generate (e.g., segment the data based on predetermined information) or may be given no instruction (e.g., allowed to generate segments based on trends present within the data). The system may further comprise applying a trained machine learning algorithm to the health dataset or one or more segmented datasets. The system may further comprise training a machine learning algorithm using the health dataset or one or more of the segmented datasets.

The system may associate a dataset of the plurality of datasets with a non-user party (530). The association may be temporary or permanent. The association may be a result of the non-user party purchasing a right to use the dataset. The right may be a license. The association may permit the non-user party to utilize the data as data for a machine learning algorithm. The association may permit the non-user party to contact at least one user of the users from whom the datasets were derived. The contacting may be with regard to establishing one or more clinical trial groups. For example, a pharmaceutical company can purchase a license to a healthcare dataset for individuals with a medical condition the company is producing a new therapeutic to treat. In this example, the pharmaceutical company can use the dataset to perform computer-based pre-trials before reaching out to the users who provided the data to recruit them for a clinical trial.

The system may transfer the dataset to the party (540). The transfer may be a digital transfer. The transfer may be permitting the party to access the data but not transferring the data from where the dataset is stored. The transfer may enable the party to apply a trained machine learning algorithm to the health dataset. The transfer may enable the party to train a machine learning algorithm using the health dataset.

### Computer Systems

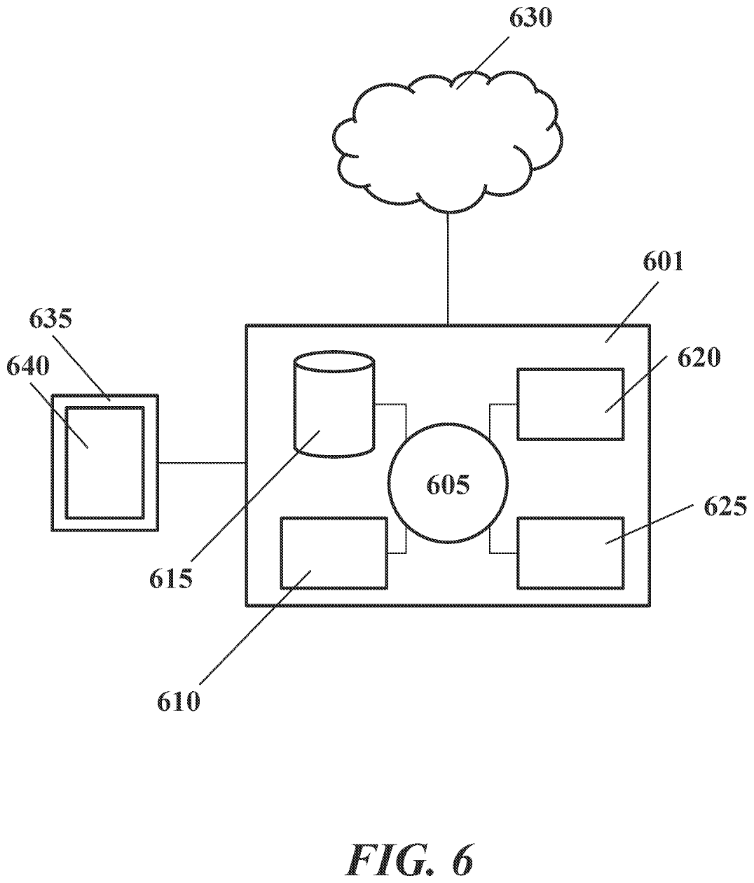

The present disclosure provides computer systems that are programmed to implement methods of the disclosure. FIG. 6 shows a computer system 601 that is programmed or otherwise configured to, for example, implement the method of FIG. 2 or FIG. 3. The computer system 601 can regulate various aspects of the present disclosure, such as, for example, the mobile healthcare application platform 400 of FIG. 4 or the health management application 100 of FIG. 1. The computer system 601 can be an electronic device of a user or a computer system that is remotely located with respect to the electronic device. The electronic device can be a mobile electronic device.

The computer system 601 includes a central processing unit (CPU, also “processor” and “computer processor” herein) 605, which can be a single core or multi core processor, or a plurality of processors for parallel processing. The computer system 601 also includes memory or memory location 610 (e.g., random-access memory, read-only memory, flash memory), electronic storage unit 615 (e.g., hard disk), communication interface 620 (e.g., network adapter) for communicating with one or more other systems, and peripheral devices 625, such as cache, other memory, data storage and/or electronic display adapters. The memory 610, storage unit 615, interface 620 and peripheral devices 625 are in communication with the CPU 605 through a communication bus (solid lines), such as a motherboard. The storage unit 615 can be a data storage unit (or data repository) for storing data. The computer system 601 can be operatively coupled to a computer network (“network”) 630 with the aid of the communication interface 620. The network 630 can be the Internet, an internet and/or extranet, or an intranet and/or extranet that is in communication with the Internet. The network 630 in some cases is a telecommunication and/or data network. The network 630 can include one or more computer servers, which can enable distributed computing, such as cloud computing. The network 630, in some cases with the aid of the computer system 601, can implement a peer-to-peer network, which may enable devices coupled to the computer system 601 to behave as a client or a server.

The CPU 605 can execute a sequence of machine-readable instructions, which can be embodied in a program or software. The instructions may be stored in a memory location, such as the memory 610. The instructions can be directed to the CPU 605, which can subsequently program or otherwise configure the CPU 605 to implement methods of the present disclosure. Examples of operations performed by the CPU 605 can include fetch, decode, execute, and writeback.

The CPU 605 can be part of a circuit, such as an integrated circuit. One or more other components of the system 601 can be included in the circuit. In some cases, the circuit is an application specific integrated circuit (ASIC).

The storage unit 615 can store files, such as drivers, libraries, and saved programs. The storage unit 615 can store user data, e.g., user preferences and user programs. The computer system 601 in some cases can include one or more additional data storage units that are external to the computer system 601, such as located on a remote server that is in communication with the computer system 601 through an intranet or the Internet.

The computer system 601 can communicate with one or more remote computer systems through the network 630. For instance, the computer system 601 can communicate with a remote computer system of a user (e.g., a smartphone, a laptop computer). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 601 via the network 630.

Methods as described herein can be implemented by way of machine (e.g., computer processor) executable code stored on an electronic storage location of the computer system 601, such as, for example, on the memory 610 or electronic storage unit 615. The machine executable or machine-readable code can be provided in the form of software. During use, the code can be executed by the processor 605. In some cases, the code can be retrieved from the storage unit 615 and stored on the memory 610 for ready access by the processor 605. In some situations, the electronic storage unit 615 can be precluded, and machine-executable instructions are stored on memory 610.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 601, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 601 can include or be in communication with an electronic display 635 that comprises a user interface (UI) 640 for providing, for example, the example user interface of FIG. 3. Examples of UI's include, without limitation, a graphical user interface (GUI), a mobile device application, and web-based user interface.

Methods and systems of the present disclosure can be implemented by way of one or more algorithms. An algorithm can be implemented by way of software upon execution by the central processing unit 605. The algorithm can, for example, be a machine learning algorithm as described elsewhere herein. The algorithm can, for example, implement the health management application or mobile healthcare application platform described herein.

Machine learning algorithms implemented on a computer or a remote server can implement methods as described elsewhere herein. For example, a machine learning algorithm can be configured to segment healthcare information. A different machine learning algorithm can be trained to manage the healthcare strategy for a user. Still another machine learning algorithm can be trained to analyze user appointment histories.

The machine learning algorithms can be supervised, semi-supervised, or unsupervised. A supervised machine learning algorithm can be trained using labeled training inputs, e.g., training inputs with known outputs. The training inputs can be provided to an untrained or partially trained version of the machine learning algorithm to generate a predicted output. The predicted output can be compared to the known output, and if there is a difference, the parameters of the machine learning algorithm can be updated. A semi-supervised machine learning algorithm can be trained using a large number of unlabeled training inputs and a small number of labeled training inputs. An unsupervised machine learning algorithm, e.g., a clustering algorithm, can find previously unknown patterns in data sets without pre-existing labels.

One example of a machine learning algorithm that can perform some of the functions described above, e.g., making classifications or segmentations of one or more health datasets, is a neural network. Neural networks can employ multiple layers of operations to predict one or more outputs, e.g., presence of a particular attribute, from one or more inputs, e.g., healthcare information of a user. Neural networks can include one or more hidden layers situated between an input layer and an output layer. The output of each layer can be used as input to another layer, e.g., the next hidden layer or the output layer. Each layer of a neural network can specify one or more transformation operations to be performed on input to the layer. Such transformation operations may be referred to as neurons. The output of a particular neuron can be a weighted sum of the inputs to the neuron, adjusted with a bias and multiplied by an activation function, e.g., a rectified linear unit (ReLU) or a sigmoid function.

Training a neural network can involve providing inputs to the untrained neural network to generate predicted outputs, comparing the predicted outputs to expected outputs, and updating the algorithm's weights and biases to account for the difference between the predicted outputs and the expected outputs. Specifically, a cost function can be used to calculate a difference between the predicted outputs and the expected outputs. By computing the derivative of the cost function with respect to the weights and biases of the network, the weights and biases can be iteratively adjusted over multiple cycles to minimize the cost function. Training can be complete when the predicted outputs satisfy a convergence condition, such as obtaining a small magnitude of calculated cost.

Convolutional neural networks (CNNs) and recurrent neural networks can be used to classify or make predictions from healthcare data. CNNs are neural networks in which neurons in some layers, called convolutional layers, receive information from small portions of a healthcare record. These small portions may be referred to as the neurons' receptive fields. Each neuron in such a convolutional layer can have the same weights. In this way, the convolutional layer can detect features, e.g., trends in a treatment efficacy, in any portion of the input health data.

RNNs, meanwhile, are neural networks with cyclical connections that can encode dependencies in time-series data, e.g., a change in a user's statues as a treatment is performed. An RNN can include an input layer that is configured to receive a series of medical records from a consistent user. An RNN can also include one or more hidden recurrent layers that maintain a state. At each time step, each hidden recurrent layer can compute an output and a next state for the layer. The next state can depend on the previous state and the current input. The state can be maintained across time and can capture dependencies in the input sequence. Such an RNN can be used to determine treatment efficacy.

One example of an RNN is a long short-term memory network (LSTM), which can be made of LSTM units. An LSTM unit can be made of a cell, an input gate, an output gate, and a forget gate. The cell can be responsible for keeping track of the dependencies between the elements in the input sequence. The input gate can control the extent to which a new value flows into the cell, the forget gate can control the extent to which a value remains in the cell, and the output gate can control the extent to which the value in the cell is used to compute the output activation of the LSTM unit. The activation function of the LSTM gate can be the logistic function.

Other examples of machine learning algorithms that can be used to process healthcare data or provide patient management are regression algorithms, decision trees, support vector machines, Bayesian networks, clustering algorithms, reinforcement learning algorithms, and the like.

The clustering algorithm can be a hierarchical clustering algorithm. A hierarchical clustering algorithm can be a clustering algorithm that clusters objects based on their proximity to other objects. For example, a hierarchical clustering algorithm can cluster users of a healthcare management platform based on the presence or absence of certain attributes, such as the user having a particular disease or receiving a specific treatment. The clustering algorithm can alternatively be a centroid-based clustering algorithm, e.g., a k-means clustering algorithm. A k-means clustering algorithm can partition n observations into k clusters, where each observation belongs to the cluster with the nearest mean. The mean can serve as a prototype for the cluster. In the context of health data, a k-means clustering algorithm can generate distinct groups of users that are correlated with each other. Thereafter, each group of users can be associated with a particular segment based on knowledge about that segment, e.g., knowledge about the users in the segment. The clustering algorithm can alternatively be a distribution-based clustering algorithm, e.g., a Gaussian mixture model or expectation maximization algorithm. Examples of other clustering algorithms that can be trained and implemented are cosine similarity algorithms, topological data analysis algorithms, and hierarchical density-based clustering of applications with noise (HDB-SCAN).

The machine learning algorithms may be configured to aid one or more workers of a medical center in managing one or more patients of the medical center who are also users of the health management application. For example, the machine learning algorithm can identify optimal dates and times for scheduling an appointment for a patient using a neural network trained on the patient's history of appointment attendance, calendar, the local weather, and the severity of the need to attend the appointment. In another example, the machine learning algorithm can notify a care manager which patients are at an increased risk of illness due to missing too many primary care physician's appointments. The machine learning algorithm may offer suggestions, reminders, alerts, or any combination thereof to the patient. For example, the machine learning algorithm can take in sensor data from the user's cell phone related to the user's activity and the user's activity history and remind the user that their doctor prescribed walking more than the user had been. In this example, the machine learning algorithm can further notify the user's doctor that the user is not being as active as prescribed.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

1. A method for providing healthcare management on a mobile electronic device of a user, comprising:
a. providing said mobile electronic device comprising a health management application comprising a plurality of health management modalities, wherein said plurality of health management modalities comprises (i) a records modality that provides a health record of said user from a health records database, (ii) an appointment modality that manages a medical appointment of said user with a medical provider with aid of a healthcare worker that is different than said medical provider and (iii) a care manager modality that facilitates communication between said user and a care manager of said user;
b. using said health management application to notify said user of said appointment; and
c. upon notifying said user, using said health management application to provide said user with one or more transit options to permit said user to attend said medical appointment.

2. The method of claim 1, further comprising, prior to (a), creating a user profile for said user.

3. The method of claim 1, wherein said mobile electronic device comprises an operating system that executes said health management application.

4. The method of claim 1, further comprising providing a one-touch emergency response system to said user.

5. The method of claim 4, wherein said one-touch emergency response system determines a location of said user with an accuracy of less than about 1 meter.

6. The method of claim 4, wherein said one-touch emergency response system transmits information to a call center.

7. The method of claim 6, wherein said call center contacts emergency medical services near said user.

8. The method of claim 6, wherein said information comprises textual information.

9. The method of claim 8, wherein said information does not comprise a voice information.

10. The method of claim 6, wherein said call center has access to said one or more health records of said user.

11. The method of claim 6, wherein said one-touch emergency response system determines a location of said user within less than about 1 second.

12. The method of claim 1, wherein said user is a recipient of healthcare from a governing body.

13. The method of claim 1, wherein said managing is managing performed by one or more people who are not said user.

14. The method of claim 1, wherein said healthcare worker is permitted to access said health record.

15. The method of claim 1, wherein said medical provider is a hospital and said healthcare worker is associated with said hospital.

16.-25. (canceled)

26. The method of claim 11, wherein said location of said user is determined by global position system (GPS) coordinates.

27. The method of claim 1, wherein said health management application allows for remote control of said mobile electronic device.

28. The method of claim 27, wherein remote control of said mobile electronic device allows a remote user to control an audio detection device an image detection device, a user input device, or any combination thereof of said mobile electronic device.

29. The method of claim 1, wherein said health management application generates an alert, and wherein said alert transmits an emergency indication to one or more other users selected by said user.

30. The method of claim 29, wherein said emergency indication comprises a time of the alert, a location of the user, information recorded with the alert, a status of the alert, medical facilities near said user, or any combination thereof.
